(19) United States
(12) Patent Application Publication
(10) Pub. No.: US 2002/0065492 A1
(43) Pub. Date: May 30, 2002

(54) Dialysis needle

(76) Inventors: James F. McGuckin JR., Radnor, PA (US); Peter W.J. Hinchliffe, Downington, PA (US); Christopher W. Maurer, Milford, CT (US)

Correspondence Address: Rex Medical, L.P., 585 County Line Road, Radnor, PA, 19087, US

(21) Appl. No.: 09/952,079

(22) Filed: Sep. 14, 2001

Related U.S. Application Data
(60) Provisional application No. 60/234,123, filed on Sep. 21, 2000.

(51) Int. Cl. A61M 31/00

(52) U.S. Cl. 604/264; 604/48

(57) ABSTRACT

An apparatus and method is provided for reducing the number of needle penetrations experienced by a dialysis patient comprising a hollow needle having penetrating tip and a fluid inlet formed in the side wall for withdrawal of blood from the patient for transport through the lumen to a dialysis machine. A tubular member is positioned inside the hollow needle and has a longitudinal lumen formed therein, terminating in a distal port, for delivery of blood to the patient received from a dialysis machine. The longitudinal lumen is isolated from the lumen of the hollow needle to prevent mixing of the withdrawn blood and the delivered blood in the apparatus. A housing retains the hollow needle and has a first chamber in fluid communication with the lumen of the hollow needle and a second chamber in fluid communication with the longitudinal lumen of the tubular member. In an alternate embodiment a separator is provided to divide the lumen of the needle into separate chambers.

This application claims priority from provisional application Serial No. 60/234,123, filed Sep. 21, 2000, the entire contents of which are incorporated herein by reference.

### BACKGROUND

1. Technical Field

This application relates to a surgical needle and more particularly to a surgical needle that transports blood to and from a patient for dialysis.

2. Background of Related Art

Hemodialysis is a well known method of simulating renal (kidney) function by circulating blood. The kidneys are organs which function to extract water and urea, mineral salts, toxins, and other waste products from the blood with filtering units called nephrons. From the nephrons the collected waste is sent to the bladder for excretion. For patients suffering from chronic renal insufficiency, hemodialysis is life saving because it provides a machine to simulate the function of the kidneys.

In the hemodialysis procedure, blood is withdrawn from the patient's body and transported to a dialysis machine, also commonly referred to as a kidney machine. In the dialysis machine, toxins and other waste products diffuse through a semi-permeable membrane into a dialysis fluid closely matching the chemical composition of the blood. The filtered blood, i.e. with the waste products removed, is then returned to the patient's body.

In one approach, an arteriovenous fistula is created so a high rate of blood flows from the artery into the patient's vein. The blood is then withdrawn directly from the patient's vein (native vein fistula) providing high rates of blood flow. This native arteriovenous fistula takes 4-6 months to develop or “mature” since it involves the vein dilating over time because of the arterial inflow. In this approach, a needle is inserted into the vein to withdraw blood from the patient for inflow to the dialysis machine and a second needle is inserted into another part of the vein to return the filtered blood from the machine to the patient. This blood exchange oftentimes needs to be done as frequently as three times each week, with the two needles remaining in the vein for up to five hours. The repeated needle punctures can eventually damage the vein beyond usability, blood clots can form and the vein can fail. Once the vein fails, it could no longer be used for access and an alternate site must be utilized.

To avoid the repetitive damage to the vein, dialysis grafts have been introduced. These grafts, typically made of PTFE, are implanted under the patient's skin, typically in the patient's forearm, and the graft is sutured at one end to the vein (venous anastomosis) for outflow and at the other end to the artery (arterial anastomosis) for inflow. This graft, which functions as a shunt creating high blood flow from the artery to the vein, enables access to the patient's blood without having to directly puncture the vein. That is, the technician sticks the two needles into the graft to respectively withdraw and return blood to the patient. The graft is also typically a loop graft to provide greater access area. The grafts are typically ready for use about four weeks after implantation. However, due to the numerous number of needle sticks because of the frequency of dialysis required for the patient, the graft may eventually deteriorate due to pseudo-aneurysm or stenosis and can no longer provide suitable access. Therefore, another surgery is required to either surgically repair the graft or implant a new graft at another site, e.g. the ipsilateral arm, contralateral arm or leg. However, since the average primary patency of the graft life is only about nine months, it is possible that eventually the surgeon will run out of suitable sites for graft implantation. Access failure could potentially be life threatening if the patient could not be hemodialyzed.

As discussed, the grafts have limited lives and must eventually be replaced by implantation of a graft at another site. One theory holds that the life of the graft is reduced by an increased number of needle sticks. This may be due to the fact that the needle sticks damage the graft wall which can cause failure. It should be noted that in most cases, the venous anastomosis downstream of the graft itself fails before the graft because of the high pressure flow, e.g. up to 1500 cc/minute, into the vein since veins by nature are designed for low pressure. However, with the advent of new graft materials to accommodate high pressure flow, the relative incidence of vein failure might be reduced and the incidence of graft failure might increase due to multiple punctures.

It would therefore be advantageous to provide a way to reduce the number of needle sticks in the graft, thereby potentially prolonging the life of the graft and consequently reducing the number of times the patient must undergo surgery to implant a new graft. Reducing the number of needle sticks in the native vein would likewise be advantageous by prolonging the life of the vein.

Furthermore, after a hemodialysis session ends, bleeding must be stopped (“hemostasis”) at the needle access puncture site. Typically patients' blood is anti-coagulated with a drug such as heparin to prevent clotting during a hemodialysis session. Thus, the time for hemostasis to occur may actually be prolonged because of the anticoagulant. Also, the loss of renal function can lead to platelet dysfunction which means the platelets are less active to form blood clots. This can also prolong the time to achieve hemostasis at the puncture site. To ensure hemostasis, the technicians typically withdraw one needle at a time at the end of the hemodialysis session, ensuring the first needle puncture is properly closed before withdrawing the second needle. Therefore, if the number of needle access sites could be reduced by half, the time to hemostasis could similarly be reduced. Also, the chances of any difficulties with closure of the puncture would also be reduced by half.

Additionally, the medical industry acknowledges that there are inherent risks to hospital staff with surgical needles. That is the staff is always concerned that an inadvertent needle stick can potentially transmit AIDS, hepatitis, or other infectious diseases. Consequently, any new device and procedure which can reduce the number of needle sticks into a patient would be greatly welcomed by the hospital staff since it would advantageously reduce the risk of inadvertent disease transmission.

### SUMMARY

The present invention provides an apparatus which advantageously reduces the number of needle sticks in dialysis patients while providing sufficiently sized lumens for transport of blood. In a first embodiment, the apparatus comprises a hollow needle having a penetrating tip at its distal end, a lumen, and a fluid inlet formed in a sidewall for withdrawal of blood from the patient for transport through the lumen. A tubular member is positioned inside the hollow needle and extends longitudinally therein. The tubular member has a longitudinal lumen formed therein, terminating in a distal port, for delivery of blood to the patient. The longitudinal lumen of the tubular member is isolated from the lumen of the hollow needle to prevent mixing of the withdrawn blood and the delivered blood in the apparatus. A central longitudinal axis of the tubular member is radially offset from a central longitudinal axis of the hollow needle.

The apparatus further includes a housing for retaining the hollow needle and has a first chamber in fluid communication with the lumen of the hollow needle and a second chamber in fluid communication with the longitudinal lumen of the tubular member.

In one embodiment, the outer wall of the tubular member is tangent to an inner wall of the hollow needle and a distalmost edge of the hollow tubular member and a distalmost edge of the hollow needle lie in the same transverse plane. Preferably, the proximal edge of the tubular member terminates proximally of the proximal edge of the needle as it extends through the housing, thereby maintaining the separation of the lumens.

In one embodiment, the tubular member is substantially circular in cross-section. In an alternate embodiment, the tubular member is substantially D-shaped in cross section, providing a smooth planar surface to reduce surface tension.

A baffle or plug, preferably in the form of glue or plastic filler material can optionally be placed between the fluid inlet (side port) and distal edge of the needle to prevent the flow of blood between the fluid inlet of the needle and the distal port of the tubular member.

In another embodiment of the present invention for reducing the number of needle penetrations experienced by a patient, an apparatus is provided comprising a hollow needle having a penetrating tip at its distal end, an interior lumen and a fluid inlet formed in the sidewall for withdrawal of blood from the patient for transport through the lumen. A separator is positioned inside the interior lumen hollow needle, extending longitudinally therein, and having first and second side edges abutting the inner wall of the needle. The separator has a width greater than an inner diameter of the needle and is bowed in a radial direction as a result of being internally stressed by contact with the inner wall of the hollow needle. The separator separates the interior lumen into first and second independent lumens. The first lumen communicates with the fluid side inlet to withdraw blood therethrough and the second lumen communicates with a distal port in the hollow needle to deliver blood to the patient. The first lumen is isolated from the second lumen to prevent mixing of the withdrawn blood and the delivered blood in the apparatus. A housing is also provided to retain the hollow needle and has a first chamber in fluid communication with the first lumen of the hollow needle and a second chamber in fluid communication with the second lumen.

In one embodiment the separator is in the form of an I-beam having first and second transverse walls abutting the inner wall of the needle and forming a seal. The transverse walls preferably each have a curvature conforming to the curvature of the interior wall.

The present invention also provides an apparatus for reducing the number of needle penetrations experienced by a patient comprising a hollow needle having a distal end, a proximal end, a sidewall, an interior lumen and a fluid inlet formed in the side wall for withdrawal of blood from the patient for transport through the interior lumen. The hollow needle has a penetrating tip to penetrate tissue and includes an axially directed slot formed at a distal end;

A shim is positioned within the slot in the hollow needle and extends longitudinally therein, separating the interior lumen into first and second independent lumens. The first lumen communicates with the fluid inlet to withdraw blood therethrough and the second lumen communicates with a distal port in the hollow needle to deliver blood to the patient. The first lumen is isolated from the second lumen to prevent mixing of the withdrawn blood and the delivered blood in the apparatus.

A housing retains the hollow needle therein and has a first chamber in fluid communication with the first lumen of the hollow needle and a second chamber in fluid communication with the second lumen.

The shim can include a distal portion with an enlarged width to help retain the shim within the slot.

### BRIEF DESCRIPTION OF THE DRAWINGS

Preferred embodiment(s) of the present disclosure are described herein with reference to the drawings wherein:

FIG. 1 schematically illustrates the dialysis needle of the present invention positioned within a dialysis graft implanted in a patient;

FIG. 2 is a top perspective view of a first embodiment of the needle of the present invention showing a shim extending beyond the proximal end of the needle;

FIG. 3A is a bottom perspective view of the needle of FIG. 2 illustrating the inlet port in the sidewall for blood inflow;

FIG. 3B is perspective view of the proximal end portion of the needle of FIG. 2 illustrating the shim extending beyond the proximal end of the needle;

FIG. 4A is a side view of the needle of FIG. 2;

FIG. 4B is a bottom view of the needle of FIG. 2;

FIG. 4C is a transverse cross-sectional view of the interior of the needle of FIG. 2, taken along an axis parallel to axis C, illustrating the shim dividing the needle interior into separate chambers;

FIGS. 5A and 5B are perspective views of a second embodiment of the dialysis needle of the present invention having a slot to receive a T-shaped shim;

FIG. 5C is an enlarged perspective view of the T-shaped shim;

FIG. 6A is a perspective view of the dialysis needle of FIGS. 5A and 5B showing the shim of FIG. 5C positioned therein;

FIG. 6B is a cross-sectional view of the needle and shim of FIG. 6A;

FIG. 7A is a transverse cross-sectional view of the interior of the dialysis needle of a third embodiment of the present invention, taken along lines A-A of FIG. 8, depicting separate chambers created by an inner tube positioned within the needle;

FIG. 7B is a perspective view of the distal end portion of the needle of FIG. 7A illustrating a baffle positioned therein;

FIG. 8 is a perspective view of the proximal end portion of the needle of FIG. 7, illustrating the inner tube extending beyond the proximal end of the needle;

FIG. 9 is a bottom view of the dialysis needle of FIG. 7 shown contained in the housing;

FIG. 10 is a side view of the dialysis needle and housing of FIGS. 7 and 9;

FIG. 11A is a top view of the dialysis needle of FIG. 7 with one of the housing halves removed to show the interior of the housing;

FIG. 11B is a top view of the dialysis needle of FIG. 7 with one of the housing halves removed to show the interior of the housing and the needle removed for clarity;

FIG. 12 is a perspective view of the proximal end portion of a fourth embodiment of the needle of the present invention depicting a D-shaped inner tube extending beyond the proximal end of the needle;

FIG. 13 is a perspective view of the proximal end portion of a fifth embodiment of the needle of the present invention depicting an I-beam separator extending beyond the proximal end of the needle;

FIG. 14a is a perspective view of the distal end portion of a sixth embodiment illustrating two tubes welded together;

FIG. 14b is an exploded view of the two tubes of FIG. 14a with the plug removed for clarity;

FIG. 15 is a transverse cross-sectional view of the two tubes taken along line 15-15 of FIG. 14a;

FIG. 16 is a longitudinal cross-sectional view of the two tubes of FIG. 14a;

FIG. 17 is a side view illustrating the distal end portion of the dialysis needle inserted within a dialysis graft for withdrawing blood from, and transporting blood to, the patient; and

FIG. 18 is a perspective view of the dialysis needle of the present invention inserted through a patch that is placed on the patient's skin to overlie the implanted graft (not shown).

### DETAILED DESCRIPTION OF PREFERRED EMBODIMENTS

Referring now in detail to the drawings where like reference numerals identify similar or like components throughout the several views, the apparatus of the present invention is designated generally by reference numeral 10. The apparatus 10 includes a hollow needle 12 and a housing 11 that allows for both the inflow and outflow of blood. More specifically, the needle 12 has two separate chambers configured so that blood can be removed from the patient's body through one chamber and returned to the patient's body through another chamber. The apparatus is specifically designed for removing blood for dialysis, although other uses are contemplated, such as plasmapheresis.

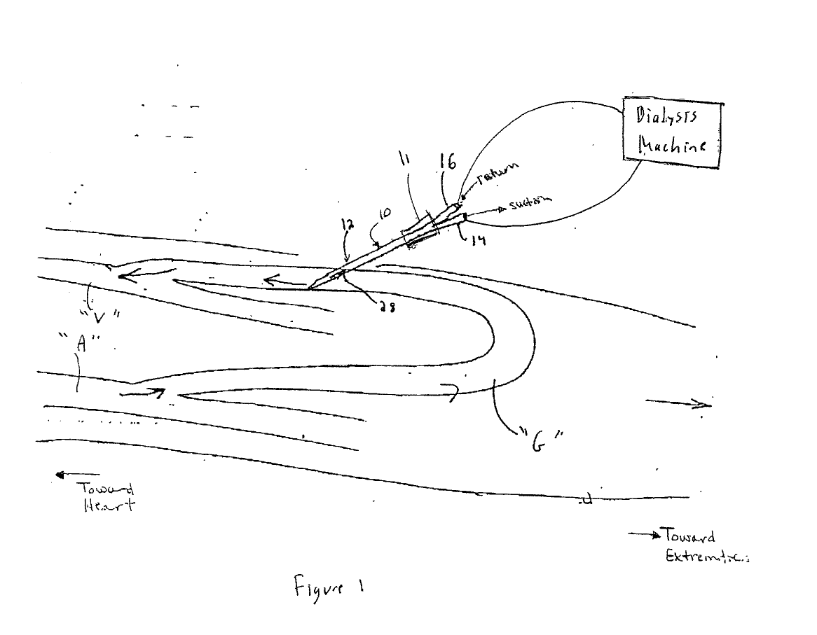

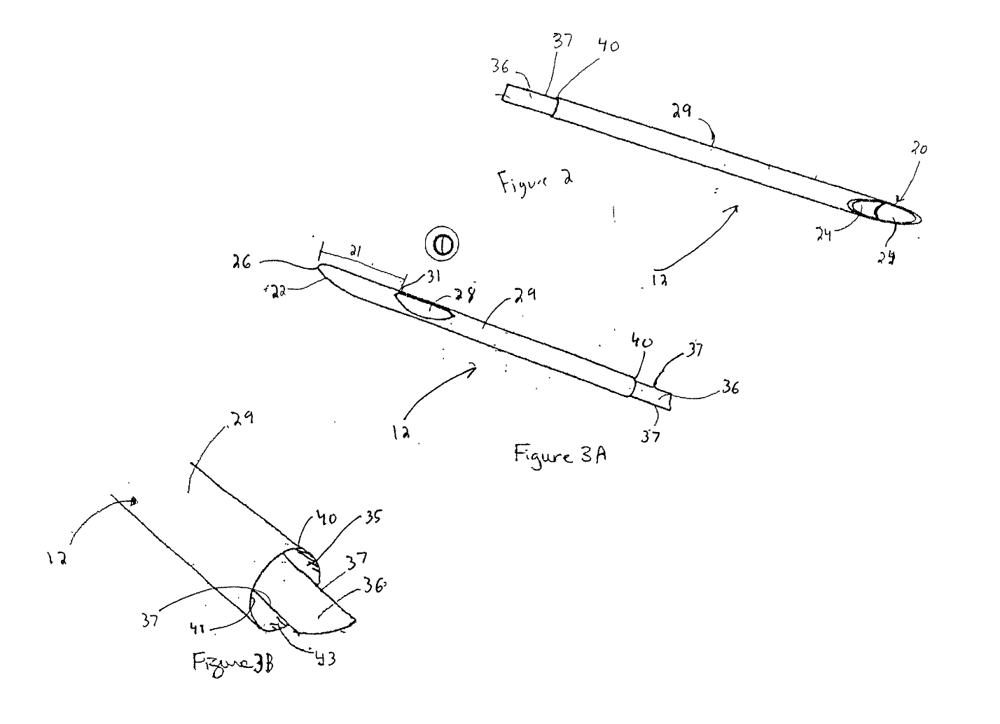

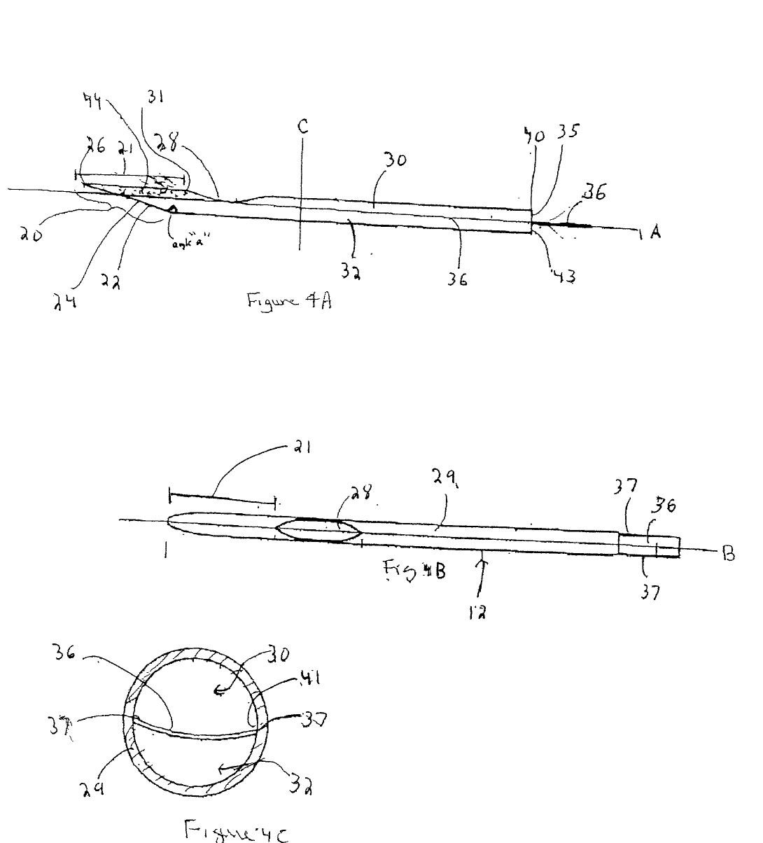

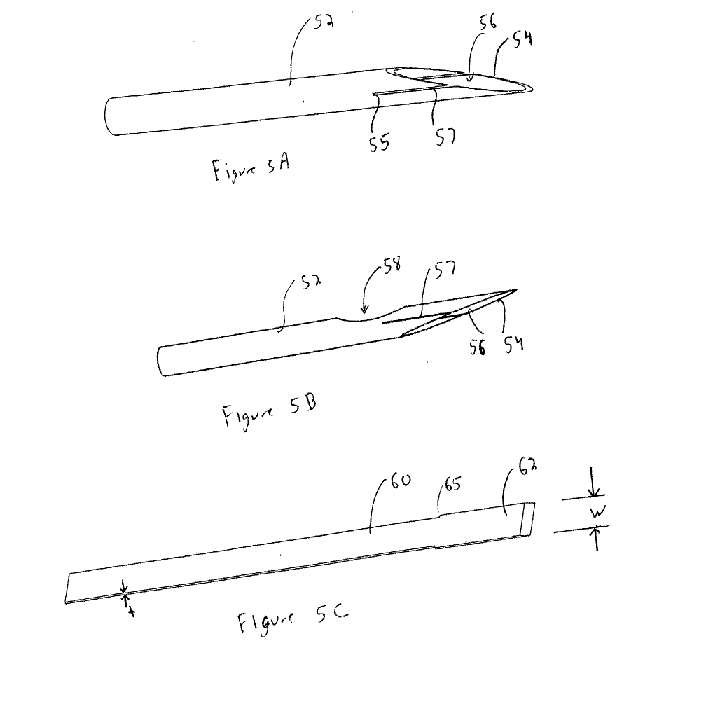

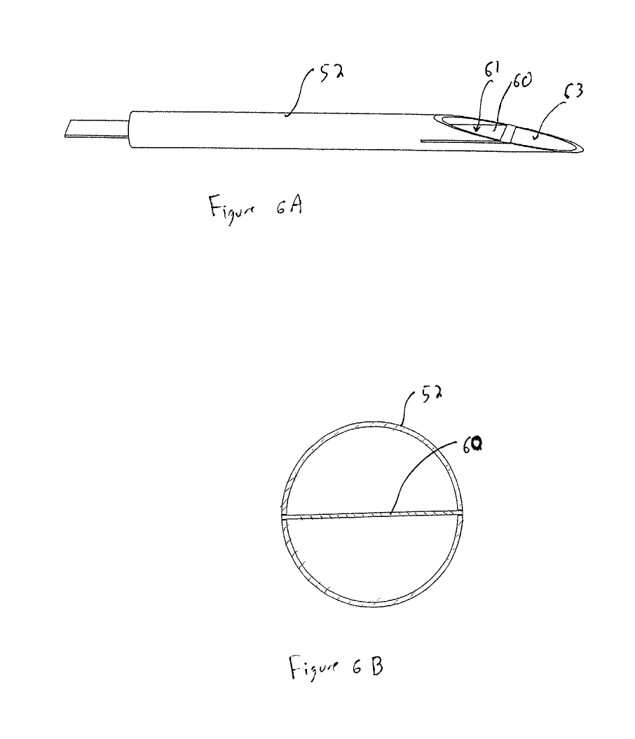

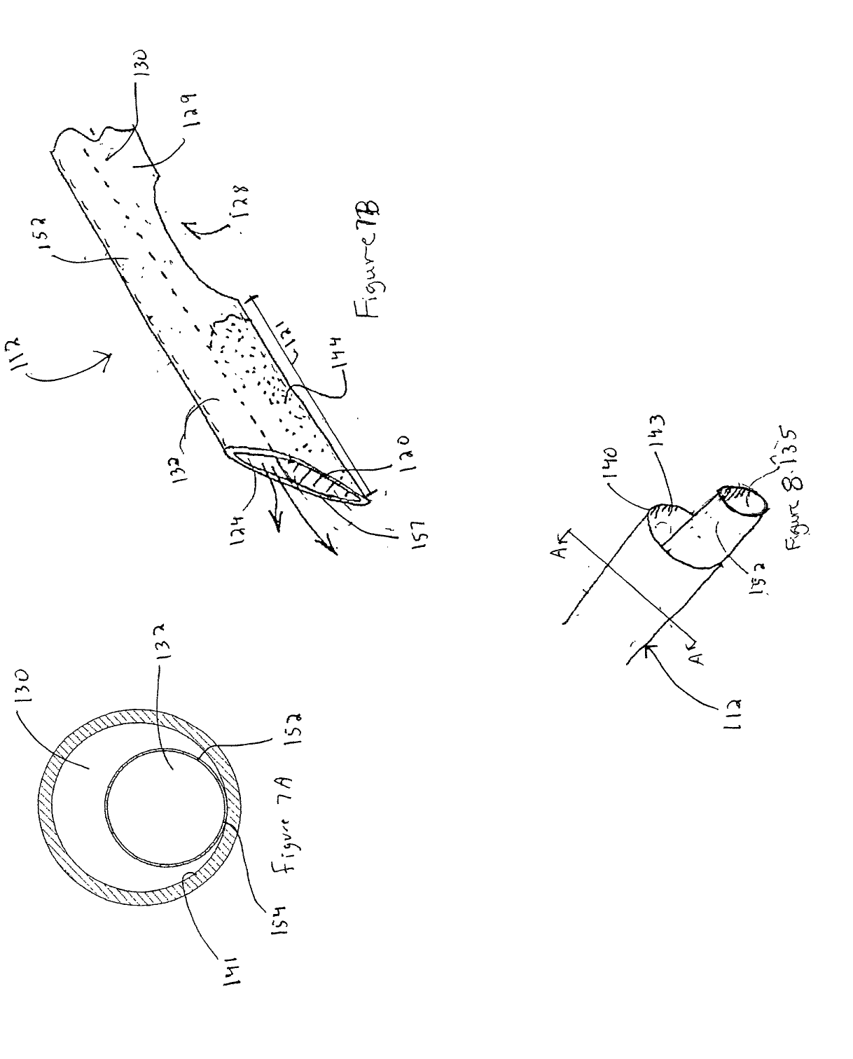

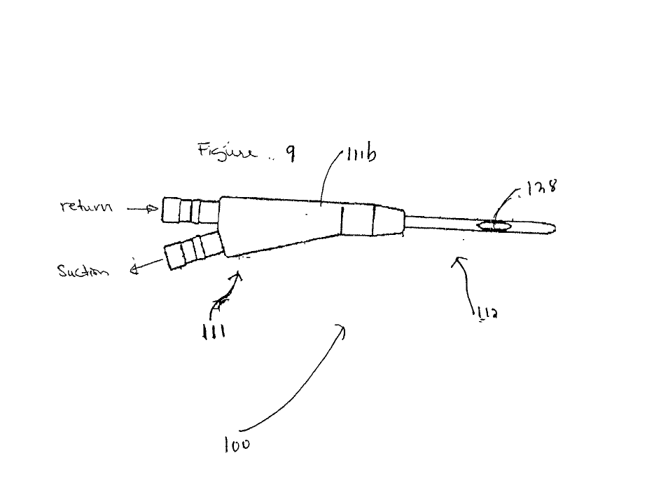

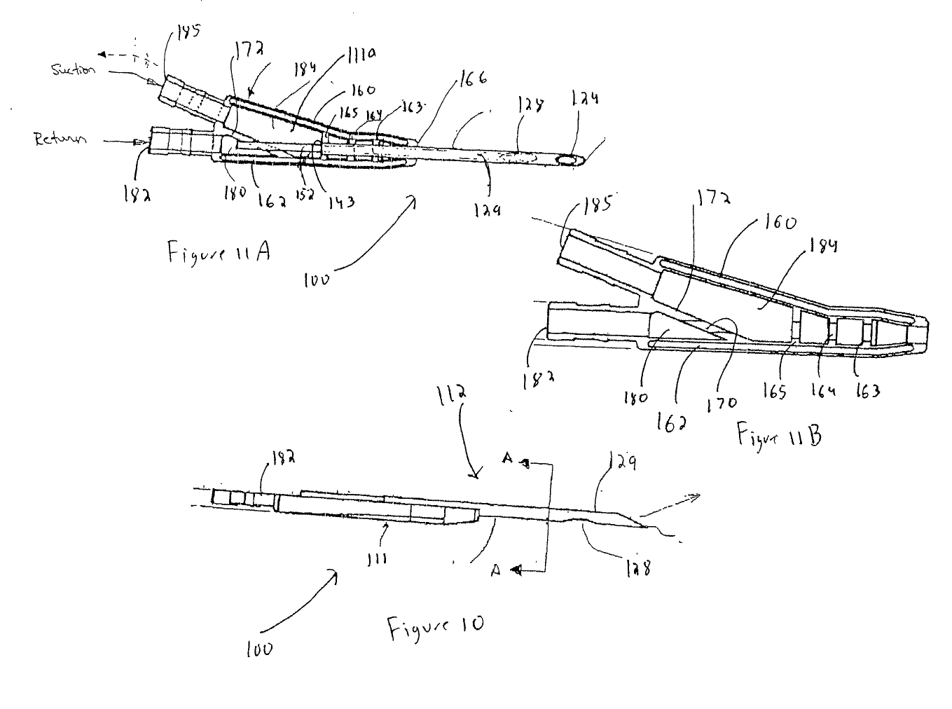

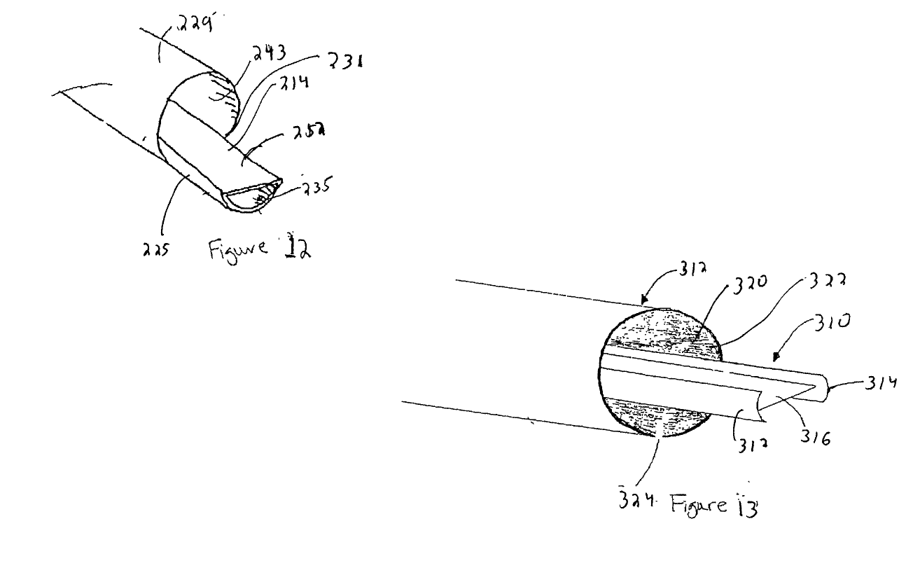

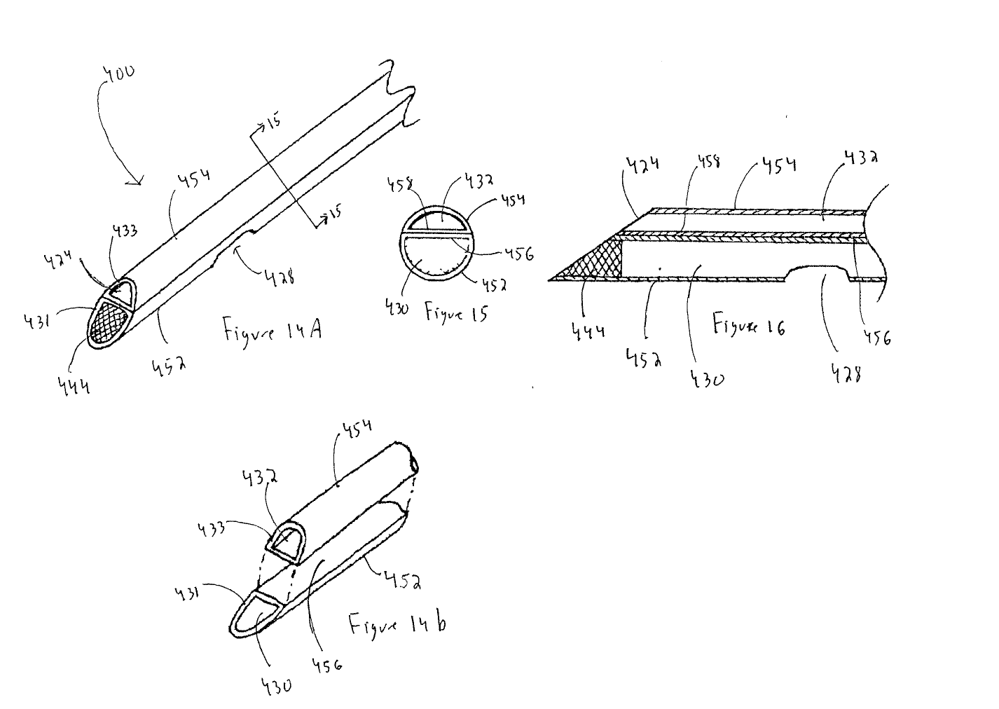

Referring to FIG. 1, apparatus 10, which provides for blood inflow and outflow by a single penetration, is shown inserted into the interior of a dialysis graft “G” mounted within a patient's arm. The graft, as is well known in the art, is surgically implanted below the patient's skin and connects the vein “V” to the artery “A”, referred to as an AV fistula, to enable blood flow from the artery to the vein, and also functioning as a shunt. The graft is shown in the conventional form of a loop to provide more surface area for needle penetration.

The apparatus 10, when inserted into the graft through a single penetration, (see FIGS. 1 and 17) enables flow of blood from the patient's body (venous flow) to a dialysis machine (shown schematically) through one chamber and enables flow of blood from the dialysis machine back to the patient through a separate chamber. The dialysis machine, as is well known, functions to filter the blood, i.e. remove wastes, of patients whose kidneys are not functioning properly. As the blood passes through the machine, toxins and other waste products diffuse through a semi-permeable membrane into a dialysis fluid closely matching the chemical composition of the blood. This removes the waste products from the blood, allowing the return of filtered blood. The outflow port of apparatus 10 which connects to tubing to transport blood to the dialysis machine is designated generally by reference numeral 14 and the inflow port which connects to tubing to transport filtered blood back to the patient is designated generally by reference numeral 16 in FIG. 1.

Referring now to the first embodiment of the apparatus illustrated in FIGS. 2-4, hollow needle 12 is illustrated. Note that the housing 11 has been removed in these drawings for convenience. Furthermore, to facilitate the description of the apparatus, needle portion 12 has been divided into three separate axes: a first longitudinal axis “A” dividing the needle into separate top and bottom sections, a second longitudinal axis “B” dividing the needle into left and right side sections; and a transverse axis “C” dividing the needle into front and back regions.

Needle 12 has a distal end 20 having a beveled tip 22, preferably at a sufficient angle “a” to facilitate penetration through the overlying tissue and graft. This angular tip also facilitates blood flow into the needle. The tip terminates in distal port 24 to deliver blood to the patient.

A fluid or side inlet (port) 28 is formed in the side wall 29 of needle 12, preferably in the bottom section, and is configured for blood intake and withdrawal. The side inlet 28 is shown substantially elliptical in shape but other shapes are also contemplated. Additionally, more than one fluid inlet could be provided.

Distal region 21 is defined for convenience as the region from distalmost tip 26 to the distal edge 31 of fluid inlet 28. Note that distal port 24 terminates distally of the distal edge 31 of fluid inlet 28. That is, the fluid inlet 28 is positioned proximally of the distal port 24 to prevent filtered blood from being withdrawn (suctioned) through inlet 28.

As can be seen in the drawings, distal port 24 is in fluid communication with chamber or lumen 32; fluid inlet 28 is in fluid communication with chamber or lumen 30. These chambers 30, 32 are independent and therefore there is no mixing of the inflow and outflow blood.

As shown in FIG. 4C, the separate chambers 30 and 32 are formed by a longitudinally extending shim 36 that is positioned inside wall 29 of needle 12. Shim 36 preferably has a width slightly greater than the inside diameter of the wall 29 to frictionally retain it therein as its edges 37 press against, and are stressed by the inner wall 41 of wall 29. Thus, when shim 36 is inserted, it is bowed in the radial direction as shown. The shim 36 extends through the entire length of the hollow needle 12, thereby creating two longitudinally extending chambers, namely chambers 30 and 32, along its entire length.

As can be appreciated, changing the width of shim 36 can alter the area of the chambers as desired since it will affect the bow. For example, in FIG. 4C, shim 36 is positioned so that chamber 30 for blood inflow is slightly greater than chamber 32 for blood outflow. However, if a larger width shim 36 is placed inside the wall 29, the bowed region would be greater (larger radius of curvature) thereby resulting in an even larger chamber 30 and smaller chamber 32.

Shim 36 is shown in FIG. 3B extending beyond the proximal edge 40 of wall 29 to maintain separation of the chambers. In this manner, when needle 12 is contained within the housing as described below, proximal inlet port 43, which is in communication with chamber 32 is isolated from proximal outlet port 35 which communicates with chamber 30.

The high exit flow velocity of the returned blood may be sufficient to prevent mixing of the blood at the distal end. However, to further ensure that inlet blood flow does not extend distally of side port 24, and that the outlet blood flow does not flow back to chamber 30, a baffle or plug 44 may optionally be provided. The baffle is preferably in the form of a glue or plastic material which plugs the distalmost region of chamber 30, i.e. the region of chamber 30 encompassing the distal region 21 as defined above. It should be appreciated that although the baffle is described and shown in the form of a material, it is also contemplated that a plastic component could be placed in the region to stanch blood flow.

FIGS. 5-6 illustrate an alternate embodiment of the dialysis needle divided into two chambers by a shim. The embodiment of FIGS. 5 and 6 is similar to the embodiment of FIGS. 2-4 in that the apparatus includes needle 52 having a beveled distal tip 54 terminating in a distal port 56. The needle 52 also has a fluid or side inlet port 58 formed in sidewall for blood intake or withdrawal. This embodiment however differs from the needle 12 of FIGS. 2-4 in the provision of shim 60 mounted within a slot 57 formed in needle 12.

More specifically, slot 57 extends axially from the distal tip 54 toward the proximal end. The slot is dimensioned to receive shim 60 which creates first and second chambers or lumens 61, 63. Shim 60 includes an enlarged width (“w”) at its distal portion 62 with shoulder 65 abutting edge 55 of slot 57, to maintain the shim 60 in place. The proximal end 64 of shim 60 is preferably insert molded for attachment at a proximal portion with a proximalmost end extending beyond the proximal end of needle 52 to maintain separation of the chambers.

The thickness “t” of shim 60 and thickness of the wall of the needle are preferably minimized in order to maximize the diameter of the chambers 61, 63. That is, the shim and needle are designed to maximize the flow area, i.e. the diameter of the chambers, while still providing sufficient structural rigidity to the needle and shim and maintaining the overall diameter of the needle at a minimum to reduce the size of the incision. In other words, a balance is struck between minimizing the overall needle size to reduce the size of the needle stick and reduce trauma to the patient and maximizing the size of the blood flow chambers to ensure sufficient blood flow to and from the patient is maintained during dialysis. This balance needs to be achieved while ensuring the components are structurally sound to withstand the blood pressure therein and penetrating forces.

The needle embodiment of FIG. 5 and 6, with the reduced thickness of the shim and needle wall, provides one way to achieve this. One example of the dimensions to achieve this is provided below. It should be understood that these dimensions are provided by way of example and other dimensions are also contemplated.

To match current flows of existing 15 gauge needles which provide blood flow in a single direction (either inflow or outflow), a 13 gauge single stick needle of the present invention can be made with an inner wall thickness of 0.005 inches and a shim of 0.002 thickness. This is demonstrated below:

Total needle area:

$$Needle diameter = .095$$

$$\begin{matrix}Area = π {r}^{2} \\ = 3.1416 \times ( .0475 ) ( .0475 ) \\ = .007088235 in\end{matrix}$$

Area of the inside of the needle:

$$Inner diameter = .90$$

$$\begin{matrix}Area = π {r}^{2} \\ = 3.1416 \times ( .0450 ) ( .045 ) \\ = .0063617725 in\end{matrix}$$

Area of the shim:

$$\begin{matrix}Area = thickness \times width \\ = .002 \times .090 \\ = .00018 in\end{matrix}$$

Since, total flow area=area of inside of needle minus area of shim, the total flow area is:

$$\begin{matrix}Area = .0063617725 in - .00018 in \\ = .006181725 in\end{matrix}$$

Note that this flow area is equivalent to the flow area of the current 15-gauge needle which has an area of 0.0031172, but can only accommodate either inflow or outflow. That is, two 15 gauge needles provide an area of 0.006234 in, that is 2×0.0031172 in. This area is substantially matched by the single stick needle of the present invention.

Another alternate embodiment of the apparatus is illustrated in FIGS. 7-11 and designated generally by reference numeral 100. The apparatus 100 functions in the same manner as apparatus 10 to transport blood to and from a dialysis patient, however, in this embodiment, the separation of the needle into two independent chambers is achieved in a different manner.

With reference first to FIGS. 7A and 8, two longitudinally extending chambers or lumens 130 and 132 are formed in hollow needle 112 by placement of inner tube 152 within lumen 132 of the needle 112. Inner tube 152, preferably composed of polyimide material, is preferably glued to inner wall 141 at its front (distal) end so its outer wall 154 is tangent to the inner wall 141 of needle 112. Inner tube 152 is preferably unattached to needle 112 proximally of the distal attachment so it can lie concentric with the needle 112 and extend through the center opening in the housing 111 when attached therein as described below.

Needle 112 has a fluid side port or inlet 128 formed therein for reception of blood from the patient. Although illustrated as elliptical, other configurations as well as additional side ports could be provided. Blood suctioned through side port 128 flows through lumen 130, exiting proximal outlet port 143 for transport to the dialysis machine. Inner tube 152 has a distal port 124. Filtered blood from the dialysis machine enters proximal inlet port 135 and flows through lumen 132 and out distal port 124 for delivery to the patient.

As shown in FIG. 7B, distal end 120 of needle 112 is beveled to facilitate penetration and blood flow in the same manner as distal tip 20 of the embodiment of FIGS. 2-4. Inner tube 152 is likewise beveled so it terminates at a distal edge coincident with the distal edge 157 of needle 112. Thus, the distal edges of the two tubes lie in the same transverse plane.

Inner tube 152, as shown in FIG. 8, extends proximally beyond the proximal edge 140 of needle 112 to maintain separation of the chambers/lumens as described below.

With reference to FIG. 7B, a baffle 144 is provided to block blood flow distal of side inlet port 128. More specifically, since side inlet port 128 is dimensioned and configured to withdraw blood from the patient's body and transport it through lumen 130 of needle 112 and must be kept separate from distal port 124 which is dimensioned and configured to deliver blood to the patient, the distal region 121 must be blocked. Baffle 144 which, like baffle 44 of the first embodiment, can be in the form of glue or plastic material or alternatively, a plastic component/s, is provided to prevent the mixing of blood in the same manner as described above as it blocks (plugs) the region below the inner tube 152 which is distal of side port 128. Since longitudinally extending chamber 130 is separate from chamber 132, and since baffle 144 prevents influx of blood while allowing blood outflow into the body, both components act concurrently to prevent mixing of inflow and outflow blood or other fluids.

As noted, inner tube 152 is dimensioned to transport blood from the dialysis machine to the patient. As shown in the cross-sectional view of FIG. 7A, the inner diameter of the outer tube 152, which is circular in cross section, preferably occupies about 60% of the total cross sectional area, with the inner diameter of the circular cross section needle 112, preferably occupying about 40%. As can be appreciated, if larger or smaller sized lumens are desired for blood inflow or outflow, a larger or smaller inner tube can be provided. Thus the cross-sectional area for blood intake and outflow can be modified without changing the outer dimensions of the apparatus 100.

Turning now to housing or casing 111 and with particular reference to FIGS. 9-11, housing 111 comprises first and second housing halves 111a and 111b that are mirror images of one another except for the tongue and groove arrangement. Housing 111b has been removed from FIGS. 10 and 11 for ease of description.

Housing 11a includes two grooves 160, 162 to receive correspondingly mating tongues of housing 111b (not shown) and the housing halves are preferably ultrasonically welded together. Semi-circular ribs 163, 164 and 165 with central slots formed therein cooperate with three identical semicircular ribs of housing half 111b to encircle and frictionally retain needle 112 within housing 111. The front end 166 of the housing is preferably sealed to prevent the egress of blood or other fluids into the housing 111. The ribs 163, 164 and 165 which have sufficient strength to support the needle, also function as a secondary, backup seal to prevent egress of any fluids that penetrate the front end seal.

Slot 170 is formed in wall 172 of housing half 111a. The proximal extension of inner tube 152 extends through this slot 170, communicating with housing chamber 180 and inflow port 182. The proximal outflow port 143 of needle 112 opens into housing chamber 184, communicating with outflow port 185. As can be appreciated, this maintains isolation of the chambers to prevent mixing of outflow and inflow blood. As should be understood, housing half 111b has an identical slot and wall which when mated with housing half 111a, complete these structures and seals.

Although the housing is shown in conjunction with the embodiment of FIG. 7, it should be appreciated that the housing 111 is also utilized with the other embodiments of the apparatus of the present invention.

Although the inner tube 252 of the embodiment of FIG. 7 is shown substantially circular in cross-section, other cross sectional configurations are also contemplated. For example, FIG. 12 illustrates one such alternative embodiment where the inner tube 252 is substantially D-shaped in cross section. This shape provides a smooth substantially planar surface 214 to reduce the surface tension and smooth the blood contact surface. Curved surfaces 225 preferably conform to the curvature of the inner wall 231 of wall 229. Proximal outlet port 243 allows for blood withdrawal and proximal inlet port 235 provides for blood inflow to inner tube 152 in a similar manner as in the embodiment of FIG. 7 described above.

FIG. 13 illustrates another alternate manner for creating two separate chambers. An I-beam 310, having curved side walls 312, 314 is positioned in the lumen 320 of the needle 312. The curved side walls 312, 314 extend transversely to the substantially planar central portion 316 of the I-beam 310 and conform to the curved inner wall 322 of needle 312, thus providing an effective seal. The I-beam functions in a similar manner to the shim 36 and shim 60 of the first and second embodiments by dividing the interior lumen 320 of the needle 312 into independent, i.e. non-communicating, chambers or lumens. The I-beam separator 310 extends beyond the proximal edge 324 of needle 312 for the same reasons as discussed above with respect to the other embodiments.

A sixth embodiment of the present invention is illustrated in FIGS. 14a-16. In this embodiment, the apparatus 400 has two tubes 452 and 454 which are either welded together or attached by other conventional means to form two separate lumens and a single penetrating tip. The D-shape tubes 452, 454 each have a planar surface 456, 458, respectively, to facilitate attachment of the tubes and minimize the overall diameter of the needle. As shown, tube 452 has a side inlet 428 communicating with a chamber or lumen 430 for blood withdrawal. Tube 454 has a distal port 424 communicating with chamber or lumen 432 for delivery of blood. The distal edges 431, 433 of both tubes 452, 454 are beveled as shown for the same reasons as discussed above. A plug or baffle 444 can be provided, as shown, to further reduce the likelihood of mixing of the inflow and outflow blood as described above. As can be appreciated, the diameter or the dimensions of the tubes 452 and 454 can be changed to alter the size of the inflow and outflow lumens.

The needle in each of the aforedescribed embodiments can be provided with a hydrophilic coating, such as heparin, on the inner wall of the chambers/lumens to increase the lubricity and to reduce clotting.

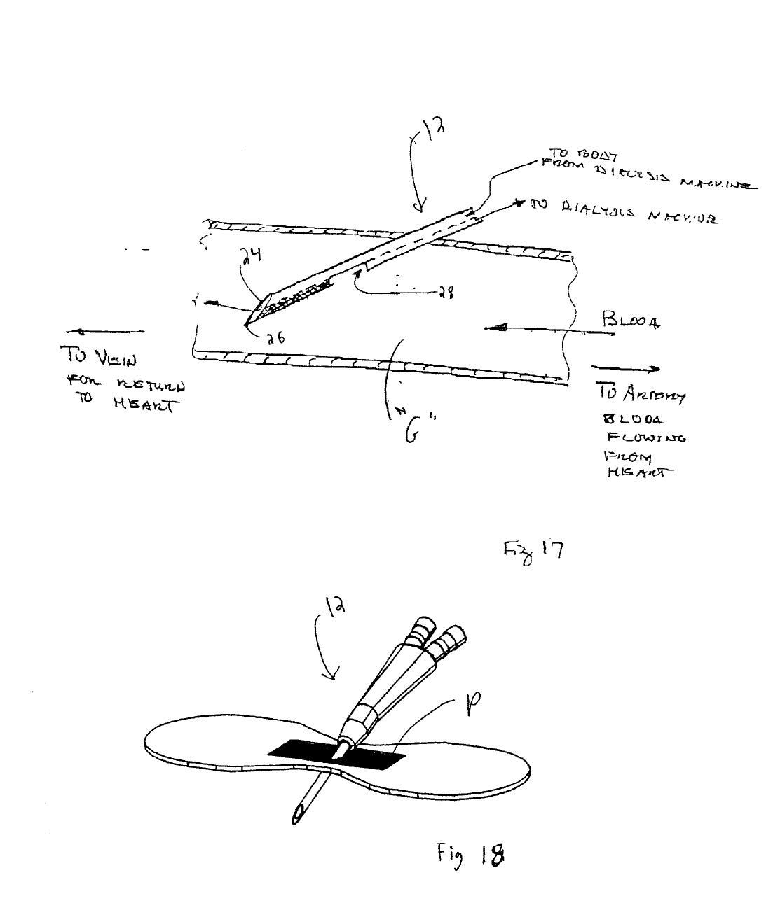

FIG. 17 illustrates the single penetration needle inserted through graft “G” to withdraw and deliver blood in the manner described in detail above. Note that the needle 12 is preferably inserted such that the tip is angled in the direction of blood flow, with the suction (fluid inlet) port 28 upstream of the distal port 24. Also, it is preferably positioned so the distalmost tip 26 points downwardly (as viewed in FIG. 17) so blood flow out of distal port 24 is directed toward the central portions of the graft G. To help hold the needle in place within the graft for the several hours required and to facilitate closure of the needle hole after withdrawal of the needle, a patch “P” can be provided as shown in FIG. 18. The patch, by keeping the hole covered, aids in post dialysis hemostasis.

While the above description contains many specifics, those specifics should not be construed as limitations on the scope of the disclosure, but merely as exemplifications of preferred embodiments thereof. For example, other structures can be provided to create separate chambers in the needle lumen. Also, the needle can also include a metal tip attached to the extruded plastic body/tubular member to enhance penetration. Those skilled in the art will envision many other possible variations that are within the scope and spirit of the disclosure as defined by the claims appended hereto.

What is claimed is:

1. An apparatus for reducing the number of needle penetrations experienced by a patient comprising:
a hollow needle having a distal end, a proximal end, a sidewall, a lumen and a fluid inlet formed in the sidewall for withdrawal of blood from the patient for transport through the lumen, the hollow needle having a penetrating tip configured to penetrate tissue;
a tubular member positioned inside the hollow needle and extending longitudinally therein, the tubular member having a longitudinal lumen formed therein, terminating in a distal port, for delivery of blood to the patient, the longitudinal lumen of the tubular member being isolated from the lumen of the hollow needle to prevent mixing of the withdrawn blood and the delivered blood in the apparatus, a central longitudinal axis of the tubular member being radially offset from a central longitudinal axis of the hollow needle; and
a housing retaining the hollow needle therein and having first and second chambers, the first chamber in fluid communication with the lumen of the hollow needle and the second chamber in fluid communication with the longitudinal lumen of the tubular member.

2. The apparatus of claim 1, wherein an outer wall of the tubular member is tangent to an inner wall of the hollow needle.

3. The apparatus of claim 2, wherein a distalmost edge of the hollow tubular member and a distalmost edge of the hollow needle lie in the same transverse plane, the plane extending transverse to the longitudinal axis of the needle.

4. The apparatus of claim 1, wherein the tubular member has a proximal edge, the proximal edge of the tubular member terminating proximally of a proximal edge of the needle.

5. The apparatus of claim 4, wherein the tubular member is substantially circular in cross section.

6. The apparatus of claim 4, wherein the tubular member is substantially D-shaped in cross section.

7. The apparatus of claim 4, further comprising a baffle within the needle to prevent the flow of blood between the fluid inlet of the needle and the distal port of the tubular member.

8. The apparatus of claim 4, wherein the first and second chambers are separated by an interior wall, the interior containing a slot to receive a proximal portion of the tubular member therethrough.

9. The apparatus of claim 1, wherein the distal tip of the needle is beveled to form the penetrating tip and a distal end of the tubular member is beveled to align with the penetrating tip of the needle to form a single penetration into the tissue of the patient for withdrawal and delivery of blood.

10. The apparatus of claim 1, wherein the housing comprises first and second housing halves when mated form a seal around the needle.

11. The apparatus of claim 1, further comprising a heparin coating within the tubular member and the needle to reduce clotting.

12. An apparatus for reducing the number of needle penetrations experienced by a patient comprising:
a hollow needle having a distal end, a proximal end, a sidewall, an interior lumen and a fluid inlet formed in the side wall for withdrawal of blood from the patient for transport through the interior lumen, the hollow needle having a penetrating tip to penetrate tissue;
a separator positioned within the hollow needle and extending longitudinally therein, the separator having first and second side edges abutting the inner wall of the needle, the separator having a width greater than the inner diameter of the needle and being bowed in the radial direction in the hollow needle as a result of being internally stressed by contact with the inner wall of the hollow needle, the separator separating the interior lumen into first and second independent lumens, the first lumen communicating with the fluid inlet to withdraw blood therethrough and the second lumen communicating with a distal port in the hollow needle to deliver blood to the patient, the first lumen being isolated from the second lumen to prevent mixing of the withdrawn blood and the delivered blood in the apparatus; and
a housing retaining the hollow needle therein and having first and second chambers, the first chamber in fluid communication with the first lumen of the hollow needle and the second chamber in fluid communication with the second lumen.

13. The apparatus of claim 12, wherein the separator is in the form of an I-beam having first and second transverse walls abutting the inner wall of the needle and forming a seal.

14. The apparatus of claim 13, wherein each of the transverse walls has a curvature conforming to a curvature of the interior wall.

15. The apparatus of claim 12, wherein the tubular member has a proximal edge, the proximal edge of the tubular member terminating proximally from a proximal edge of the needle.

16. The apparatus of claim 12, further comprising a baffle within the needle to prevent the flow of blood between the fluid inlet and the distal port.

17. An apparatus for reducing the number of needle penetrations experienced by a patient comprising:
a hollow needle having a distal end, a proximal end, a sidewall, an interior lumen and a fluid inlet formed in the side wall for withdrawal of blood from the patient for transport through the interior lumen, the hollow needle having a penetrating tip to penetrate tissue, the hollow needle including an axially directed slot formed at a distal end;
a shim positioned within the slot in the hollow needle and extending longitudinally therein, the shim separating the interior lumen into first and second independent lumens, the first lumen communicating with the fluid inlet to withdraw blood therethrough and the second lumen communicating with a distal port in the hollow needle to deliver blood to the patient, the first lumen being isolated from the second lumen to prevent mixing of the withdrawn blood and the delivered blood in the apparatus; and
a housing retaining the hollow needle therein and having first and second chambers, the first chamber in fluid communication with the first lumen of the hollow needle and the second chamber in fluid communication with the second lumen.

18. The apparatus of claim 17, wherein the shim has a distal portion with an enlarged width to help retain the shim within the slot.

19. An apparatus for reducing the number of needle penetrations experienced by a patient comprising:
a needle having first and second tubular members, the first tubular member having a distal end, a proximal end, a side wall, a lumen and a fluid inlet formed in the side wall for withdrawal of blood from the patient for transport through the lumen, the tubular member being non-circular in cross-section and having a first penetrating tip configured to penetrate tissue;
the second tubular member positioned alongside the first tubular member, the first tubular member having a longitudinal lumen formed therein, terminating in a distal port, for delivery of blood to the patient, the longitudinal lumen of the tubular member being isolated from the lumen of the first tubular member to prevent mixing of the withdrawn blood and the delivered blood in the apparatus, the second tubular member being non-circular in cross section and having a second penetrating tip aligned with the first penetrating tip so together the first and second penetrating tips form a single penetration in the tissue for withdrawing and delivering blood.

20. The apparatus of claim 19, further comprising a housing retaining the first and second tubular members therein and having first and second chambers, the first chamber in fluid communication with the lumen of the first tubular member and the second chamber in fluid communication with the longitudinal lumen of the second tubular member.
